(19) United States
(12) Patent Application Publication
(10) Pub. No.: US 2010/0125229 A1
(43) Pub. Date: May 20, 2010

(54) Controllable Joint Brace

(75) Inventors: Katherine Rudolph, Wilmington, DE (US); Jian-Qiao Sun, Merced, CA (US)

Correspondence Address: RATNERPRESTIA, P.O. BOX 1596, WILMINGTON, DE, 19899, US

(73) Assignee: University of Delaware, Newark (DE)

(21) Appl. No.: 12/501,176

(22) Filed: Jul. 10, 2009

Related U.S. Application Data
(60) Provisional application No. 61/079,921, filed on Jul. 11, 2008.

(51) Int. Cl. A61F 5/00; A61B 5/103

(52) U.S. Cl. 602/16; 600/595

(57) ABSTRACT

A controllable brace assembly includes a thigh brace having a thigh gyroscope attached thereto and a shank brace having a shank gyroscope and a shank accelerometer attached thereto. A controllable knee lock couples the thigh brace to the shank brace. The knee lock has an encoder attached thereto. At least one pressure sensor is adapted to be attached to a shoe. A controller is configured to receive signals from the thigh gyroscope, the shank gyroscope, the encoder, the shank accelerometer, and the at least one pressure sensor. The controller transmits a first signal set to engage or disengage the controllable knee lock based on the received signals. A method of controlling the brace is also disclosed.

### CROSS REFERENCE TO RELATED APPLICATIONS

This application claims priority to U.S. provisional application No. 61/079,921, filed Jul. 11, 2008, which is incorporated herein, in its entirety, by reference.

### STATEMENT REGARDING FEDERALLY SPONSORED RESEARCH

The U.S. Government has a paid-up license in this invention and the right in limited circumstances to require the patent owner to license others on reasonable terms as provided for by the terms of NIH Grant # R21 HD047468, awarded by the National Institutes of Health.

### FIELD OF THE INVENTION

The present invention relates to a knee, foot, ankle orthosis that automatically adjusts locking and unlocking the knee joint based on the gait of the user.

### BACKGROUND OF THE INVENTION

Typically during gait training in people with profound lower limb weakness, the knee of the weak limb is locked to prevent collapse when the weak limb supports body weight during the stance phase. Because the knee is locked, it cannot bend normally when the leg swings forward in preparation for the next step. The inability to advance the limb normally requires the individual to develop secondary compensations to advance the weak leg. The secondary compensations then become ingrained in the walking pattern and may need to be addressed in future rehabilitation.

### SUMMARY OF THE INVENTION

Briefly, the present invention provides a controllable brace assembly comprising a thigh brace having a thigh gyroscope attached thereto and a shank brace having a shank gyroscope and a shank accelerometer attached thereto. A controllable knee lock couples the thigh brace to the shank brace. The knee lock has an encoder attached thereto. At least one pressure sensor attaches to the sole of a user's shoe. A controller is configured to receive signals from the thigh gyroscope, the shank gyroscope, the gyroscope, the encoder, the shank accelerometer, and the at least one pressure sensor and to transmit a first signal set to engage or disengage the controllable knee lock based on the received signals.

Further, the present invention provides a controllable brace assembly comprising a knee, ankle, foot orthosis comprising a knee lock, a plurality of sensors coupled to the knee, ankle, foot orthosis, and a first lock configured to releasably lock the knee lock based on data provided by the plurality of sensors.

The invention also provides methods of operating the controllable brace assembly.

### BRIEF DESCRIPTION OF THE DRAWINGS

The invention is best understood from the following detailed description when read in connection with the accompanying drawings. It is emphasized that, according to common practice, various features/elements of the drawings may not be drawn to scale. On the contrary, the dimensions of the various features/elements may be arbitrarily expanded or reduced for clarity. Moreover, in the drawings, common numerical references are used to represent like features/elements. Included in the drawing are the following figures:

FIG. 1 is a perspective view of a knee brace according to an exemplary embodiment of the invention, coupled to a user;

FIG. 2 is a side elevational view of a knee joint of the knee brace shown in FIG. 1;

FIG. 3 is a graph illustrating knee flexion angle during a typical gait cycle;

FIG. 4 is a side elevational view of the knee brace of FIG. 1 coupled to the user;

FIG. 5 is a front elevational view of the knee brace of FIG. 1 coupled to the user;

FIG. 6 is a front elevational view of the knee joint of the knee brace of FIG. 1;

FIG. 7 is a perspective view of the lower thigh portion and the upper shank portion of the knee brace of FIG. 1, illustrating the location of sensors on the outer and rear aspect of the brace;

FIG. 8 is a bottom plan view of a shoe incorporating four pairs of sensors used in the knee brace of FIG. 1;

FIG. 9 is an electrical schematic illustrating an exemplary solenoid driving circuit and a pressure sensor signal processing circuit used with the knee brace of FIG. 1;

FIG. 10 is a schematic drawing of an exemplary computer integration configuration with the knee brace of FIG. 1;

FIG. 11 is an exemplary screen display illustrating locking strategies and locking angles used to control operation of the knee brace of FIG. 1;

FIG. 12 is an exemplary screen display illustrating data obtained using the knee brace of FIG. 1;

FIG. 13 is an exemplary model of real time gait event detection and lock control using the knee brace of FIG. 1;

FIG. 14 is an exemplary model of a subsystem that determines stance and swing based on pressure readings using the knee brace of FIG. 1;

FIG. 15 is a graph showing moving statistics of an encoder used with the knee brace of FIG. 1;

FIG. 16 is an exemplary model of a subsystem that calculates various statistical models based on gyroscope and accelerometer data using the knee brace of FIG. 1;

FIG. 17 is an exemplary model of a lock control subsystem that uses results of gait event detection using the knee brace of FIG. 1;

FIG. 18 is a graph illustrating kinematic data from sensors on the knee brace of FIG. 1 for a healthy person;

FIG. 19 is a graph illustrating kinematic data from sensors on the knee brace of FIG. 1 for a stroke victim;

FIG. 20 is a graph illustrating correlation between kinematic data, moving statistics from horizontal acceleration, and initial contact and toe off events using the knee brace of FIG. 1; and

FIG. 21 is an exemplary model of a detection subsystem that uses statistical values along with raw signal data to detect initial contact and toe off using the knee brace of FIG. 1.

### DETAILED DESCRIPTION OF THE INVENTION

Certain terminology is used herein for convenience only and is not to be taken as a limitation on the present invention. The terminology includes the words specifically mentioned, derivatives thereof and words of similar import. The following describes exemplary embodiments of the invention. However, it should be understood based on this disclosure, that the invention is not limited by the exemplary embodiments of the invention.

Referring to the Figures in general, a brace assembly 100 according to an exemplary embodiment of the present invention includes two computer controlled locks that are used in rehabilitating walking in persons with lower extremity weakness, such as stroke victims.

Brace assembly 100 is programmed to limit the bending and straightening of a user's knee while walking. Brace assembly 100 incorporates locks that can be activated in the stance and/or swing phases of gait, depending on the needs of the user and the goals of the gait training activities. Brace controls allow nearly immediate adjustments to the locking strategies to allow a user to attempt to control his/her walking patterns without assistance of Brace assembly 100 for as little as one stride of walking. Allowing a user to attempt to control the limb independently allows the user to experience error in his/her walking pattern, which is vital to motor learning. The flexibility of the locking strategies also makes brace assembly 100 a useful research tool that allows investigation of a myriad of research studies involving neural control of walking and balance.

A feature of brace assembly 100 is the ability to lock the knee at any prescribed angle during one part of the gait cycle, while allowing movement in the remainder of the gait cycle. The locking characteristics mitigate the development of secondary compensations and would hasten the development of more normal movement patterns and higher functional walking. Using this application, the brace technology may be useful in the rehabilitation of people with muscle weakness and or reduced lower limb sensation such as after stroke, traumatic brain injury or degenerative musculoskeletal conditions.

The ability of brace assembly 100 to influence leg motion during walking has many other uses than simply a brace to control knee motion. One application of brace assembly 100 may be in improving balance responses in people with balance deficits. When the knee is prevented from moving normally, the normal equilibrium of the walking pattern is disturbed. The disturbance will influence the balance of the individual requiring a reaction to regain equilibrium and continue walking. Brace assembly 100 has the capability of disturbing balance in a myriad of ways during the swing phase and the stance phase. The locking strategies produce balance disturbances across a spectrum from a mild disturbance to one that causes a fall. The computer controls are ideally suited to apply and track body motion so that the lock angle and timing can be adjusted to disturb balance enough to challenge users but not enough to elicit a fall. Computer algorithms may continually adjust the behavior of brace assembly 100 to match that of the user for optimal training.

Brace assembly 100 may also be used as an assistive device (rather than a training device). For example, people with leg weakness may use a wheelchair for their primary mode of locomotion but when in the community they might need assistance walking. Brace assembly 100 may assist such persons.

The primary advantage of brace assembly 100 is the ability to program the locks to engage when desired. As a training tool, brace assembly 100 allows real-time adjustments to walking patterns as users practice walking within a single treatment session or as they progress in walking over weeks of rehabilitation.

Brace assembly 100 according to the present invention can be programmed to respond almost immediately, within one to two strides of walking, to user behavior. The immediate adjustment to user behavior allows therapists to incorporate principles of motor learning to rehabilitation interventions. For example, the users' gait pattern can be assisted with brace assembly 100 for a bout of training, and then brace assembly 100 can be turned off, thereby allowing users to attempt the movement on their own. Errors associated with the attempt to move on their own are vital to motor learning and the immediate adjustments to controls on the knee brace are uniquely suited for such an application.

Brace assembly 100 is a knee brace with two computer controlled locks that may be used in rehabilitating walking in persons with lower extremity weakness such as stroke. Brace assembly 100 is programmable to limit the user's knee from bending or straightening during walking. The locks can be activated in the stance and/or swing phases of gait depending on the needs of the user and the goals of the gait training activities. The controls of brace assembly 100 allow nearly immediate adjustments to the locking strategies to allow users to attempt to control their walking patterns without assistance of brace assembly 100 for as little as one or two strides of walking.

Allowing users to attempt to control their limb independently allows them to experience error in their walking pattern, which is vital to motor learning. The flexibility of the locking strategies also makes brace assembly 100 a useful research tool that allows investigation of a myriad of research studies involving neural control of of walking and balance.

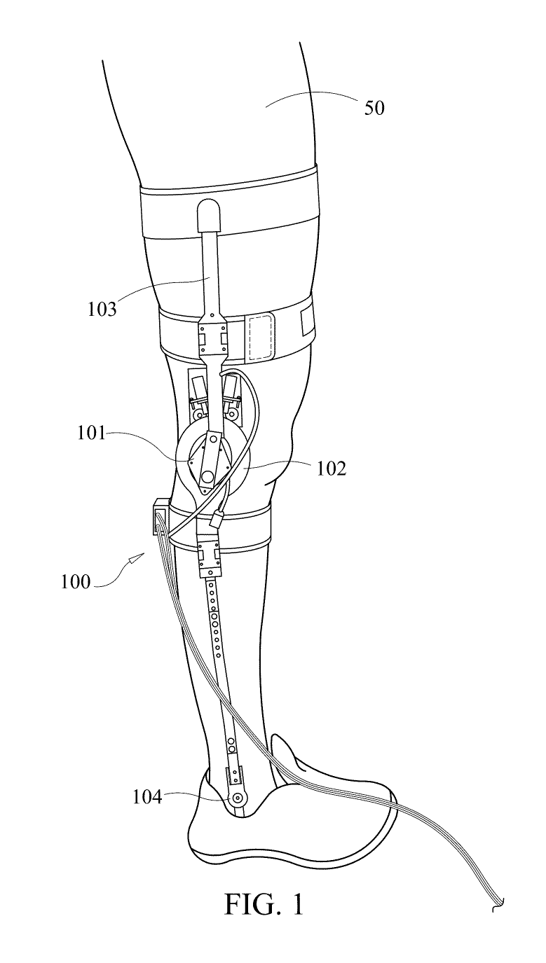

An exemplary embodiment of the inventive brace assembly 100 on a user 50, shown in FIG. 1, consists of a standard knee, ankle, foot orthosis (KAFO) 101 with single axis joints 102, 104 at the knee and ankle, respectively. The total range of motion provided by brace assembly 100 is 110° flexion to 25° hyper-extension.

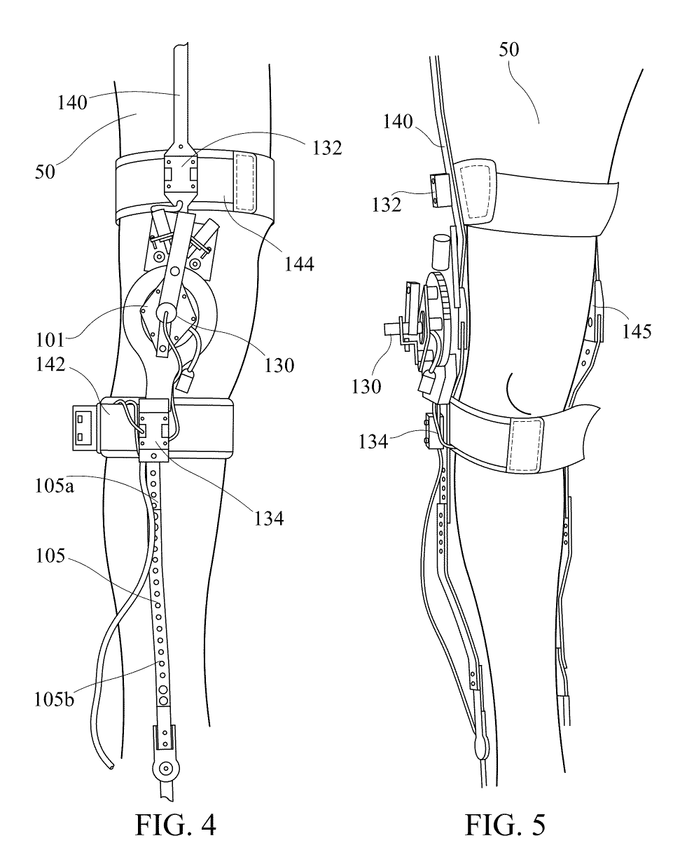

KAFO 101 includes a thigh portion 103 and a shank portion 105 (illustrated in FIG. 4) that is constructed from an upper shank portion 105a and a lower shank portion 105b. The height of shank 105 is adjustable via screw holes along at least a portion of the upper shank portion 105a and lower shank portion 105b, which may be aligned and coupled together by nuts/bolts to form shank 105 of a desired length.

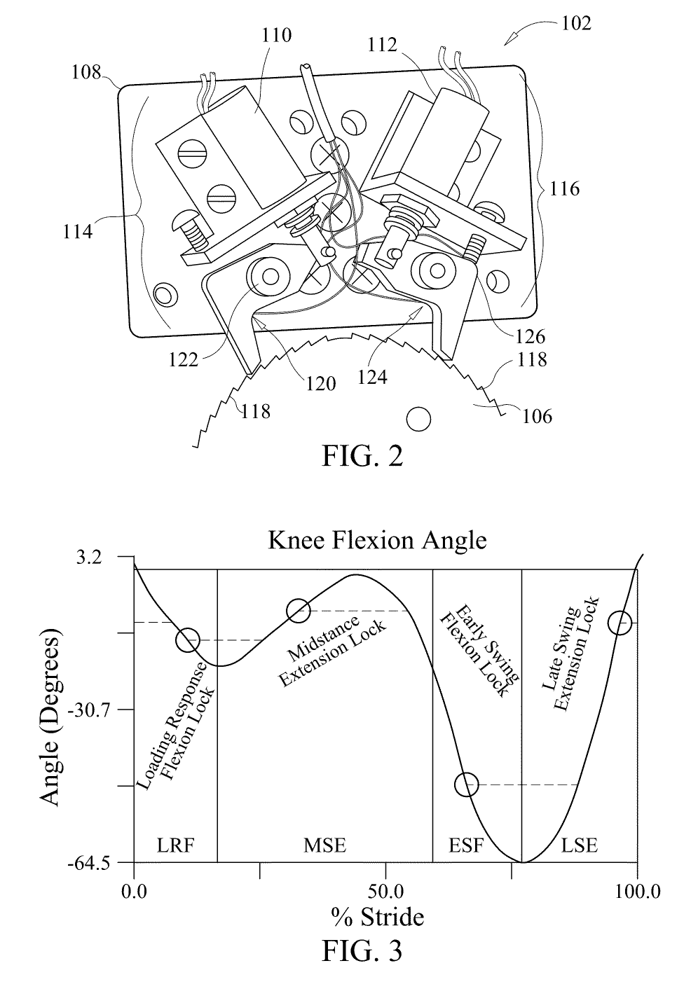

Referring to FIG. 2, knee joint 102 includes a locking mechanism including a gear plate 106 and a base plate 108 for anchoring a pair of solenoids 110, 112 to selectively engage gear plate 106 for locking knee joint 102 and disengaging gear plate 106 for allowing knee joint 102 to rotate.

Base plate 108 is fitted with two locks 114, 116, a first lock 114 to limit flexion and a second lock 116 to limit extension. The total range of motion provided by brace assembly 100 is about 110° flexion to about 25° hyper-extension.

Gear plate 106 has gear teeth 118 spaced at predetermined angles along the outer circumference of gear plate. In an exemplary embodiment, gear teeth 118 are spaced at 5 degree increments. Gear teeth 118 are spaced around outer circumference over an arc of about 135 degrees, which is sufficient for locking knee joint 102 in both flexion and extension directions.

A first locking pawl 120 is pivotally coupled to base plate 108 about a first pivot 122. First locking pawl 120 releasably engages gear teeth 118 to releasably lock knee joint 102 from moving in a flexion direction. First solenoid 110 engages pawl 120 with gear teeth 118 based on input from control software as prescribed by a programmer, such as a physical therapist. Pawl 120 engages gear teeth 118 at knee joint 102, thus preventing rotation in the flexion direction, but rides over gear teeth 118 when rotating in the extension direction.

A second locking pawl 124 is pivotally coupled to base plate 108 about a second pivot 126. Second locking pawl 124 releasably engages gear teeth 118 to releasably lock knee joint 102 from moving in an extension direction. Second solenoid 112 engages pawl 124 with gear teeth 118 based on input from control software as prescribed by a programmer, such as a physical therapist. Pawl 124 engages gear teeth 118 as knee joint 102 rotates in the extension direction, but rides over gear teeth 118 thus allowing movement in the flexion direction.

Solenoids 110, 112 may be programmed to engage and disengage their respective pawls 120, 124 during the course of a full stride. A graph illustrating engagement of locks 114, 116 during a full stride is shown in FIG. 3. The stride may be broken into four (4) phases as follows:
1. Flexion lock in stance (LRF)—knee is prevented from flexing beyond prescribed angle (pawl 120 engaged) during stance phase, the knee will extend freely;
2. Extension Lock in Stance (MSE)—knee is prevented from extending beyond prescribed angle (pawl 124 engaged) during stance phase but can flex freely;
3. Flexion Lock in Swing (ESF)—knee is prevented from flexing beyond prescribed angle (pawl 120 engaged) during swing phase but the knee can extend freely; and
4. Extension Lock in Swing (LSE)—knee is prevented from extending beyond prescribed angle (pawl 124 engaged) during the swing phase but can flex freely.

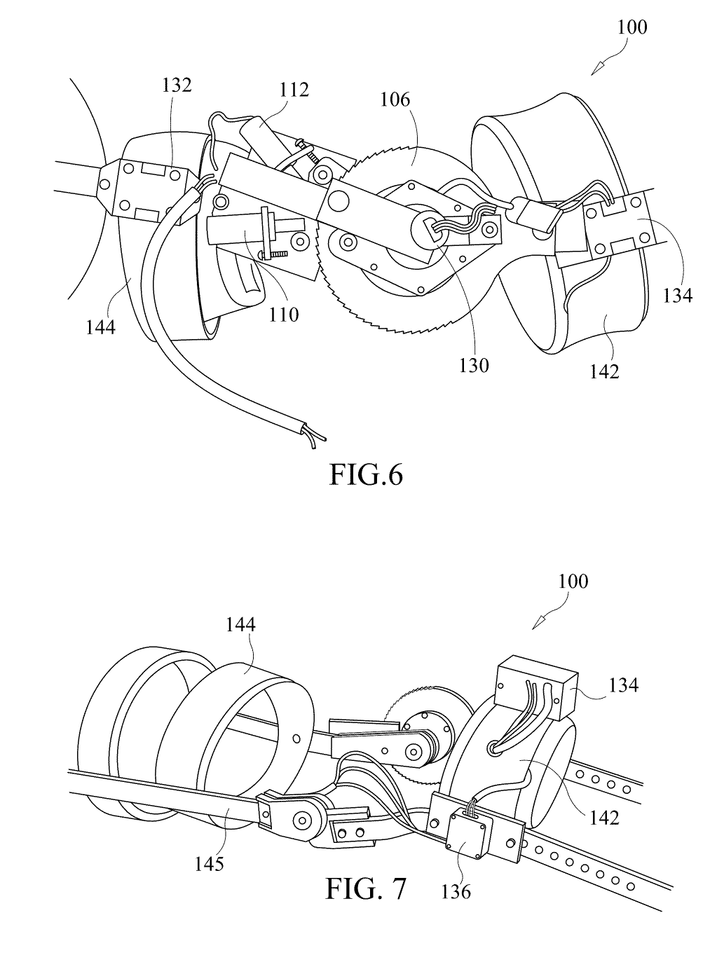

Referring to FIGS. 4-8, a sensor array that is disposed about brace assembly 100 provides data to determine gait characteristics of user 50 as well as relative positions of KAFO 101 during the user's stride. Sensor array includes a magnetic encoder 130, thigh and shank gyroscopes 132, 134, respectively, a shank accelerometer 136, and a plurality of pressure sensors 138 that all receive data inputs from the surrounding environment and transmit the data to a processor.

A miniature absolute magnetic shaft encoder 130 detects knee motion. An exemplary encoder may be a Model MA2 encoder, manufactured by US Digital of Vancouver, Wash.

Two gyroscopes 132, 134 rated at 300°/sec are mounted to the lateral upright 140 of KAFO 101; a shank gyroscope 134 is at the level of shank cuff 142 and a thigh gyroscope 132 is at the level of a thigh cuff 144. An exemplary gyroscope 132, 134 may be a Model ADXRS300EB, manufactured by Analog Devices of Norwood, Mass. Gyroscopes 132, 134 measure the angular velocity about their own centers and are mounted in the same plane with each other. The difference in the two signals is the knee joint angular velocity and is highly useful in a Gait Event Detection (GED) algorithm.

A biaxial accelerometer 136, rated at 5 g, is mounted to a medial upright 145 of KAFO 101 at the level of shank cuff 142. An exemplary accelerometer 136 may be a Model ADXL320, manufactured by Analog Devices of Norwood, Mass. Accelerometer 136 provides horizontal and vertical acceleration of brace assembly 100 in the sagittal plane. The accelerometer data are highly insensitive to the movement of the limb within brace assembly 100. The signals generated by accelerometer 136 are used in the GED algorithm.

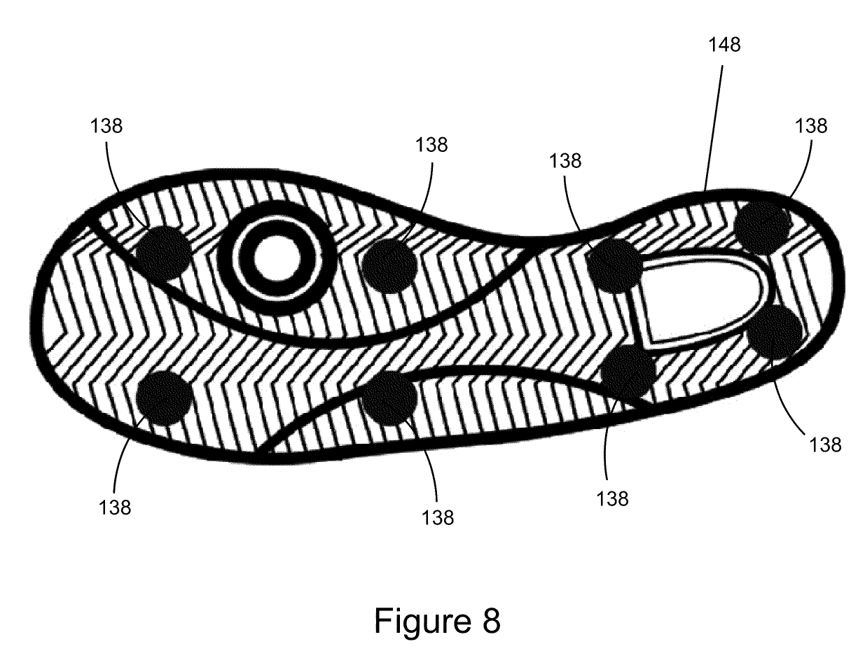

Referring now to FIG. 8, four pairs of pressure sensors 138 are placed on the sole of the user's shoe 148 to detect stance and swing phases of gait. Exemplary sensors 138 may be the Flexiforce A201, manufactured by Tekscan of Boston, Mass. The force range of the pressure sensors is 0-25 lb.

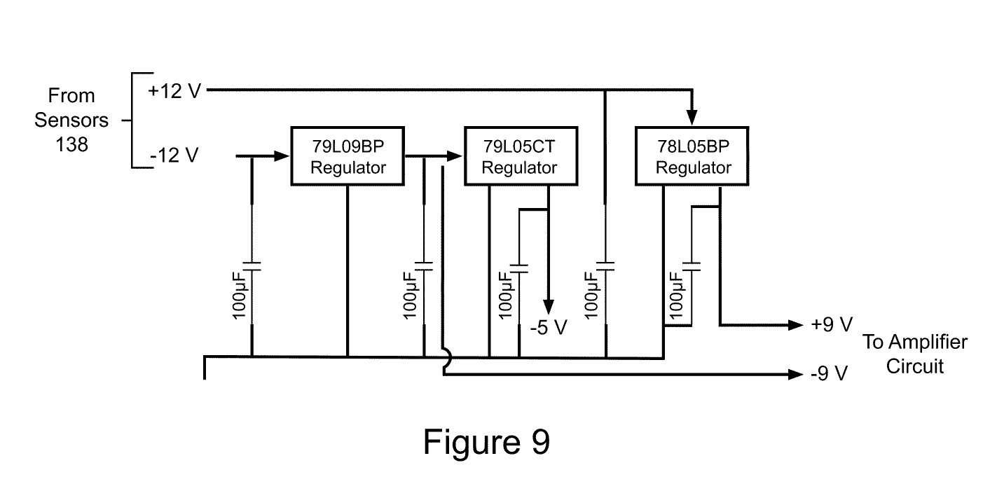

Sensors 138 are constructed of two layers of substrate (polyester/polyimide) film. On each layer, a conductive material (silver) is applied, followed by a layer of pressure-sensitive ink. Adhesive is then used to laminate the two layers of substrate together to form sensor 138. With no load on sensor 138, the resistance between the two conductive layers is extremely high. As the load is increased, the resistance decreases. A supply signal is applied to a lead connected to one of the layers and the output signal increases due to the decreased resistance. As shown in FIG. 9, an electrical circuit used with pressure sensors 138 serves three purposes: supply voltage, signal addition, and amplification. The resulting voltage is used to detect stance or swing phase of gait and sends signals to solenoids 110, 112 to engage or disengage the lock pawls 120, 124.

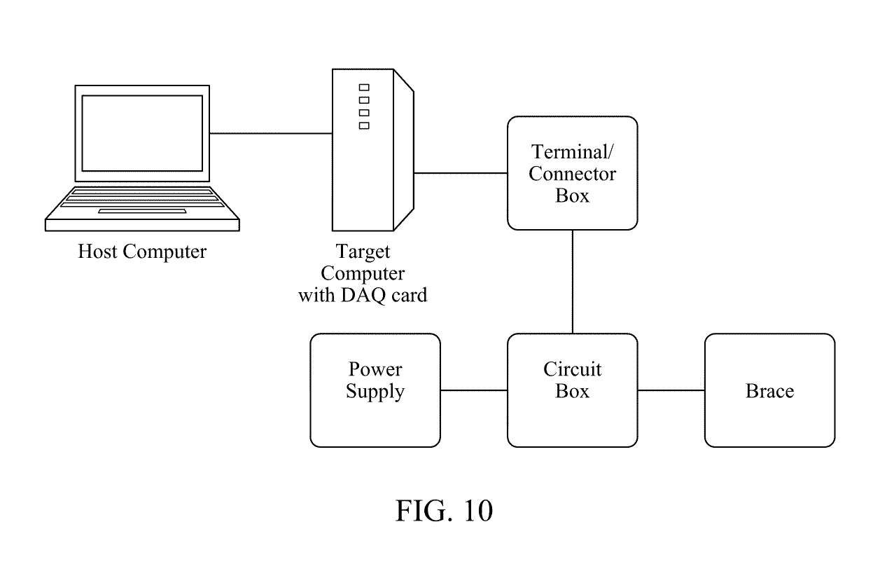

Referring to FIG. 10, a graphic user interface (GUI) 200 allows operators to input lock strategies, lock durations and lock angles as well as view data as it is being collected from brace assembly 100. A controller in a target computer 201 receives data from and transmits signals to brace assembly 100, thus allowing rapid control of locks 114, 116. More specifically, the controller receives signals from thigh gyroscope 132, shank gyroscope 130, encoder 134, shank accelerometer 136, and pressure sensors 138 and transmits a signal set to engage or disengage locks 114, 116 on knee joint 102 based on the received signals.

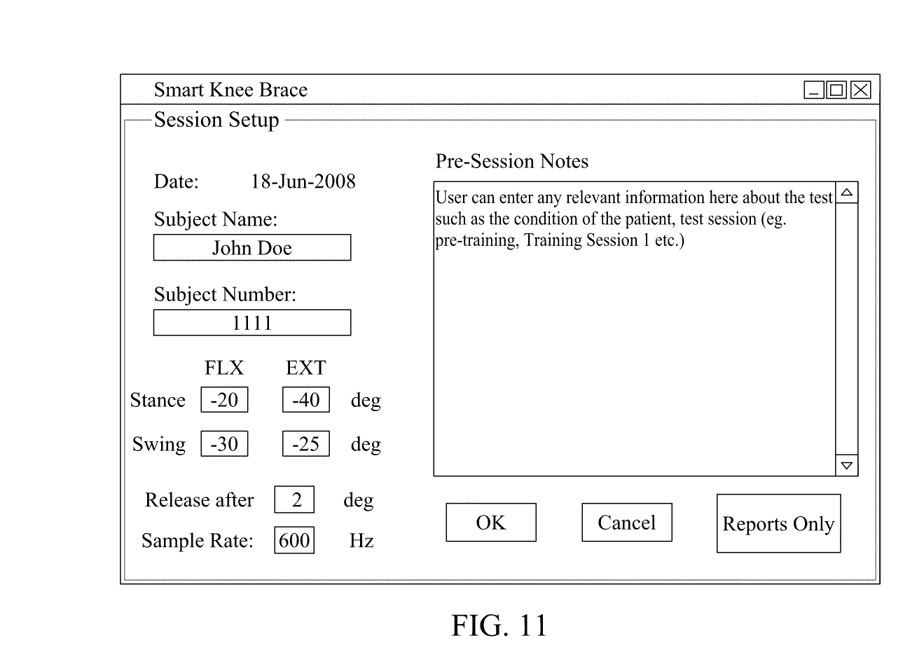

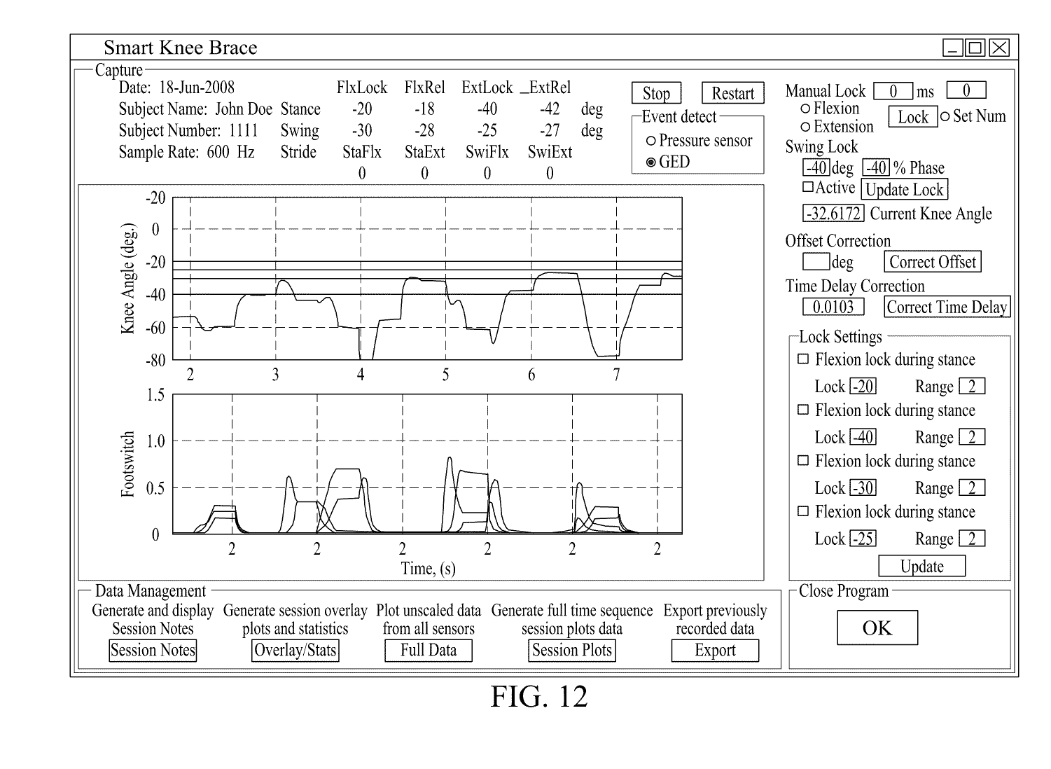

Several screens may be used to input notes before the data collection session is started and to control the brace during its use. A Session Setup Screen 202 is shown in FIG. 11. Screen 202 allows an operator, such as a therapist, to program operating parameters for brace assembly 100, such as during a therapy session. A Data Capture Screen 204, shown in FIG. 12, represents operating data received from the sensor array. Upper graph represents knee angle, with lock thresholds. Lower graph represents signals received from each of the four pairs of pressure sensors 138.

Proper function of brace assembly 100 depends on accurate methods to detect the events of the gait cycle. Brace assembly 100 has two (2) methods for detecting gait events: (1) pressure sensors 138 that detect stance and swing and (2) a gait event detection (GED) algorithm that detects initial contact (IC) and toe off (TO) during walking in a Simulink model based primarily on data from shank and thigh gyroscopes 132, 134 and the horizontal acceleration from shank mounted accelerometer 134. Two methods for detecting gait events allow reliable detection in users with widely varying gait patterns.

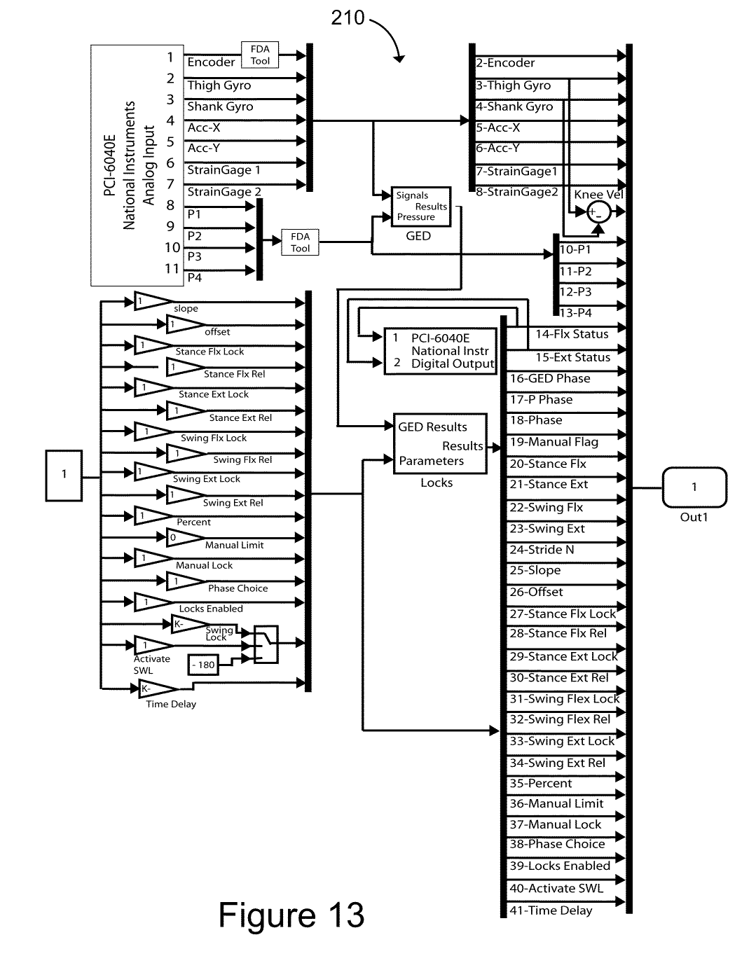

The knee angle signal coming from magnetic encoder 130 may not necessarily be reliable for detecting gait events and knee angle data is used only for reporting purposes. Data from gyroscopes 132, 134 and accelerometer signal 136 are extremely reliable and consistent even among different users. Once the data are captured, the data is used to setup windows in time in which events will be detected and used to control solenoids 110, 112. A Simulink model 210 for real time gait event detection and control is illustrated in FIG. 13. This model outputs the current phase of the gait cycle.

One of the most powerful aspects of the current event detection methods is constant calibration of the values used in the detection of events. Thus, as user 50 slows down or speeds up over a training session the events will be adjusted within two (2) strides. That is, the system is configured to transmit first signal set based on initial system parameters and then change to transmit a second signal set, different from the first signal set, to engage or disengage the pawls 120, 124 based on the received signals.

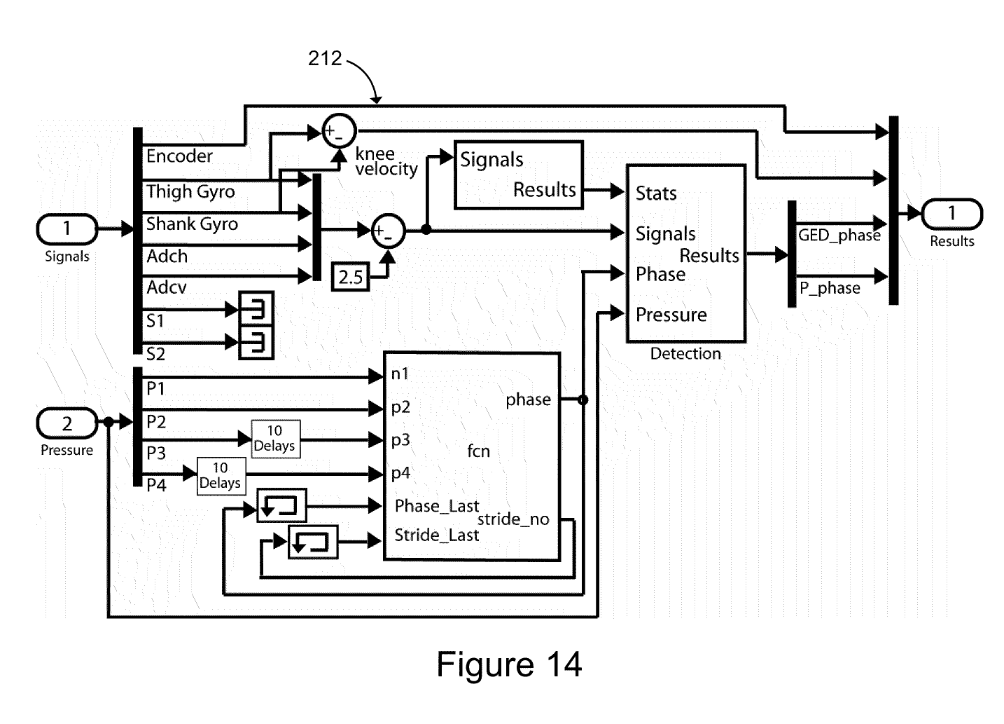

When set to use pressure sensors 138, an embedded Matlab function 212, shown in FIG. 14, in the GED subsystem determines the phase of the gait using simple threshold values from the different pressure sensors 138. If sensors 138 under the foot are reading pressure above the threshold (read as sensors P1, P2, P3, P4 in FIG. 14), then user 50 is in stance and if the pressure is subthreshold, then user 50 is in swing.

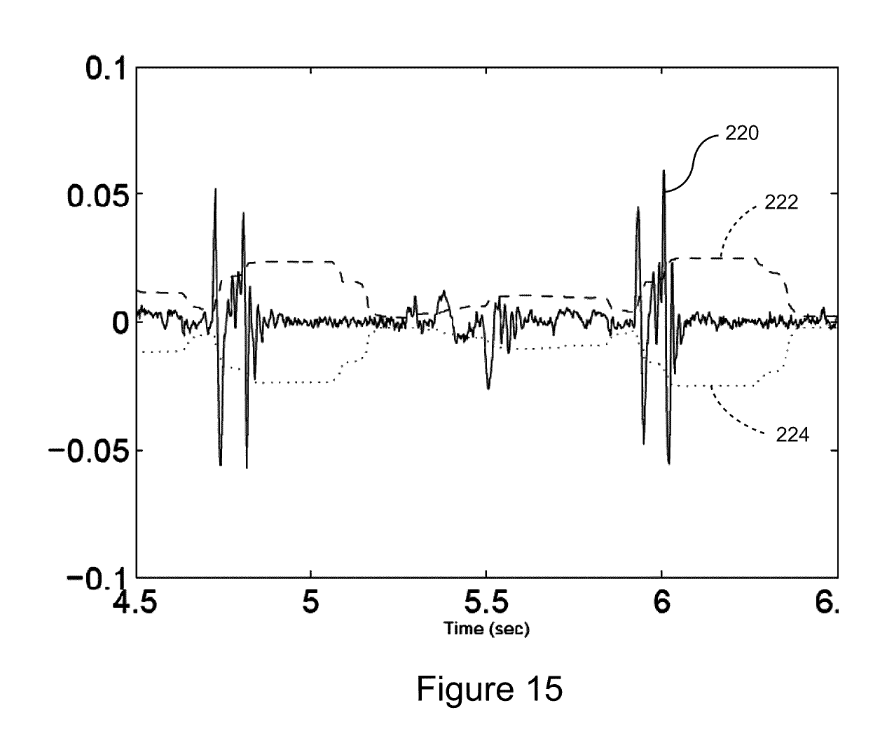

When set to use the GED, data from gyroscopes 132, 134 and accelerometer 136 are used to determine the events of initial contact by the foot on the ground and when the toe comes off the ground. The Stats Subsystem's primary goal is to calculate statistical values of the accelerometer and gyroscope signals that will be used in detecting gait events. Three of the statistical outputs are moving statistics shown in FIG. 15. A moving average of the slope of the horizontal encoder is shown as a solid line 220. An upper standard deviation bound is shown as the upper dashed line 222 and a lower standard deviation bound is shown as the lower dashed line 224. The standard deviation bounds are 1.5 times the actual standard deviation of the moving average signal.

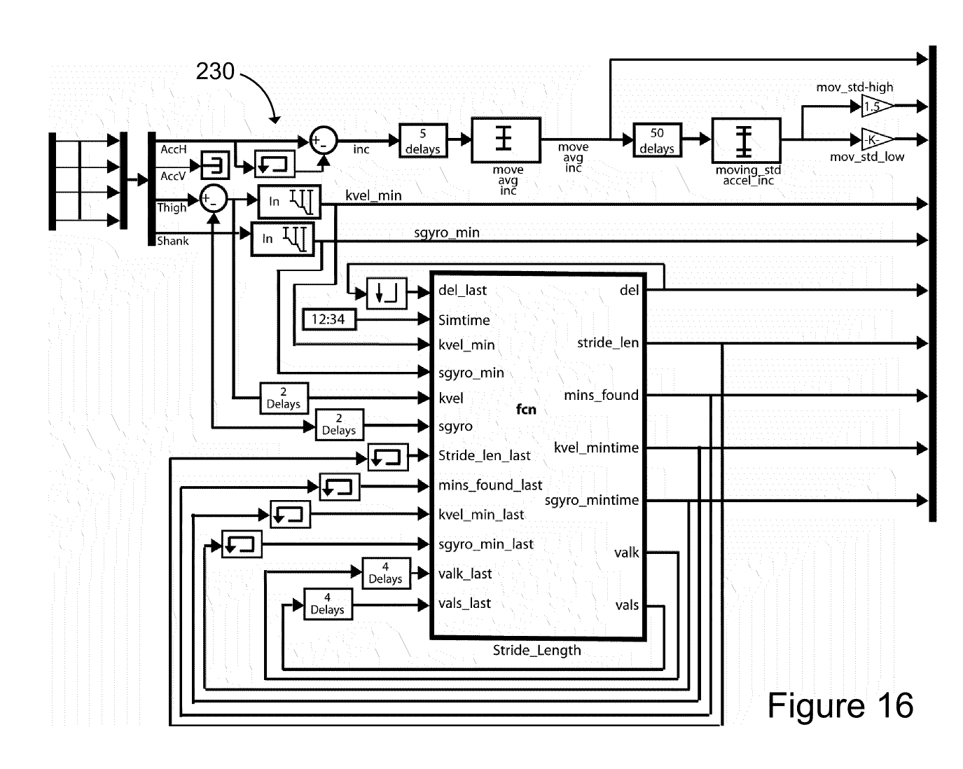

A Stats Subsystem Simulink model 230, shown in FIG. 16, calculates various statistical values based on the gyroscope and accelerometer data that are used to detect initial contact and toe off. Model 230 determines a moving average of the slope from the horizontal signal of accelerometer 136 and the standard deviation of this moving average using the equations below.

In the equations below, aₕ represents the horizontal acceleration signal, aₚ the calculated moving average over n past points, and s(j) the moving standard deviation. These are both simple calculations for which predefined Simulink blocks from the Statistics Blockset are used.

$$\begin{matrix}{a}_{p} = \frac{1}{n} {\sum }_{i = j - n + 1}^{j} [ {a}_{h} ( 1 ) - {a}_{h} ( i - 1 ) ] , & Equation 1 \\ \overline{{a}_{p}} = \frac{1}{n} {\sum }_{i = jn + 1}^{j} {a}_{p} , & Equation 2 \\ s ( j ) = [ \frac{1}{n} \sum {( {a}_{p} ( i ) - {( {a}_{p} ( i ) - \overline{{a}_{p}} )}_{30}^{2} ]}^{\frac{1}{2}} & Equation 3\end{matrix}$$

Upper and lower standard deviation bound are found by multiplying s(j) by ±1.5 respectively. These high and low values simply form boundaries to help in the detection of initial contact in conjunction with the moving average. The moving average and the upper and lower standard deviation bounds found here are the primary signals utilized for detecting initial contact. A general trend of these moving statistics is shown in FIG. 15. The data displayed in FIG. 15 was from a healthy user wearing brace assembly 100.

The slope of the horizontal accelerometer signal is used because there is a sharp spike in the signal at initial contact that is highly consistent in healthy and stroke users. In essence, the algorithm does not necessarily look for the amplitude of the accelerometer signal, but the slope of the signal spike. When detected inside the correct timing window, the moving average from above traveling outside of the standard deviation bounds is a highly reliable indicator of initial contact. The 1.5 factor has been determined from experimentation with various sets of healthy and stroke user data.

Referring back to FIG. 16, the next two critical statistics to be determined are the knee velocity and shank gyro moving minimums. These values are reliable and consistent and are used in the determination of timing windows as well as for calculating stride length. Predefined Simulink blocks track the running minimums of these signals. The values are output from the Stats Subsystem 230 of FIG. 16 as well as input into the embedded Matlab function 212 of FIG. 14 that determines the time based stride length.

The complete set of outputs from the embedded stride length function include “del” (a measure of time between a shank gyro minimum and the previous knee velocity minimum), “stride_len” (the stride length in seconds), “mins_found” (the number of shank gyro minimums found), “kvel_mintime” (the time of the last shank gyro minimum and the time of the last knee velocity minimum). The knee velocity is used to track the stride duration, time of knee velocity minimums, and for finding a minimum count used to track the number of strides taken. There are several criteria that must be met for these values to be updated. The code path that is used on the knee velocity signal is shown below. In this process, Time is the current simulation time, KVel_min_last is the time of the last noted knee velocity minimum, and Stride_Len is the time in seconds of the last stride. The difference between the knee velocity minimum and the current knee velocity value is taken at each time step. The four most recent values calculated make up the variable Valk. KVel contains the two most recent knee velocity values and KVelCrit (an internal variable of the program, which is the maximum knee joint velocity allowed, and is set by the user) is an updating value that is a percentage of the knee velocity minimum that must be reached before the knee velocity signal can be considered as a new minimum. Mins_Found is a count of the knee velocity minimums that have been detected and Kvel_mintime is the time of the new knee velocity minimum that has been detected. The major outcomes of this are the updated stride length, minimums found count, and the updated time of the last knee velocity minimum.

If more than one minimum in the knee velocity has been found, similar criteria are also checked against the shank gyroscope signal instead of the knee velocity signal. This is used to update the time of the most recent shank gyroscope minimum which is used in the time windowing for the actual event detection. In the case that a new shank gyroscope minimum is found, the variable del is updated to reflect the time elapsed between the last knee velocity minimum and the new shank gyroscope minimum.

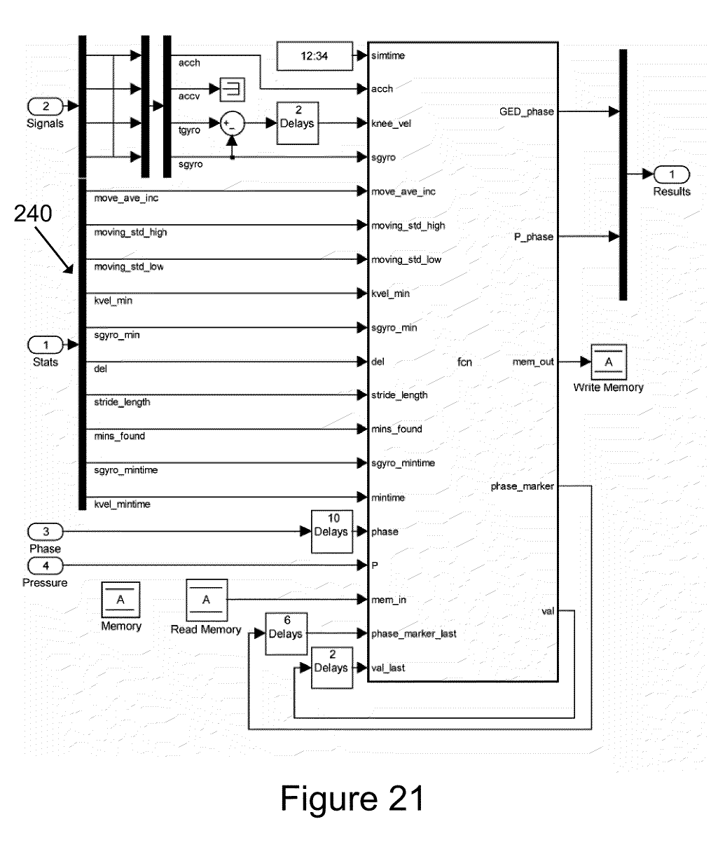

The Detection Subsystem 240 (shown in FIG. 21) uses inputs from the Stats Subsystem 230, the raw data signals from gyroscopes 132, 134 and accelerometer 136, the phase signal generated from pressure sensors 138, and the raw signals from pressure sensors 138 themselves to detect gait events and output a final phase signal to be used in control of locks 114, 116. A major task of this subsystem 240 is to properly time window the signals. Time windowing defines a specific amount of time for which a particular event can be checked. For instance, while there may be many times that the moving average of the accelerometer slope exceeds the set standard deviation bounds (see FIG. 15), the correct one that identifies IC only occurs during a specific time window.

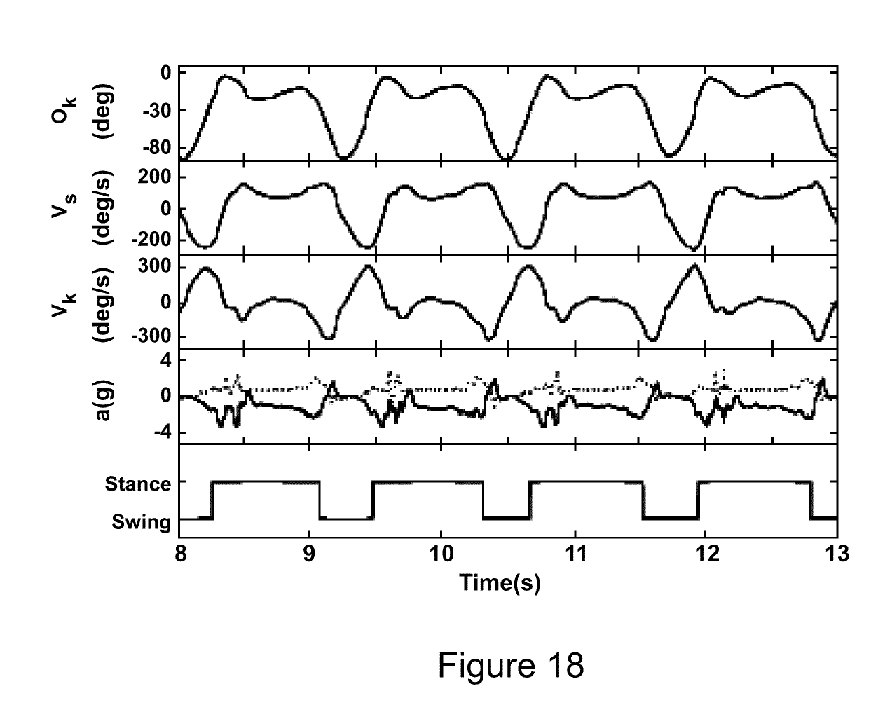

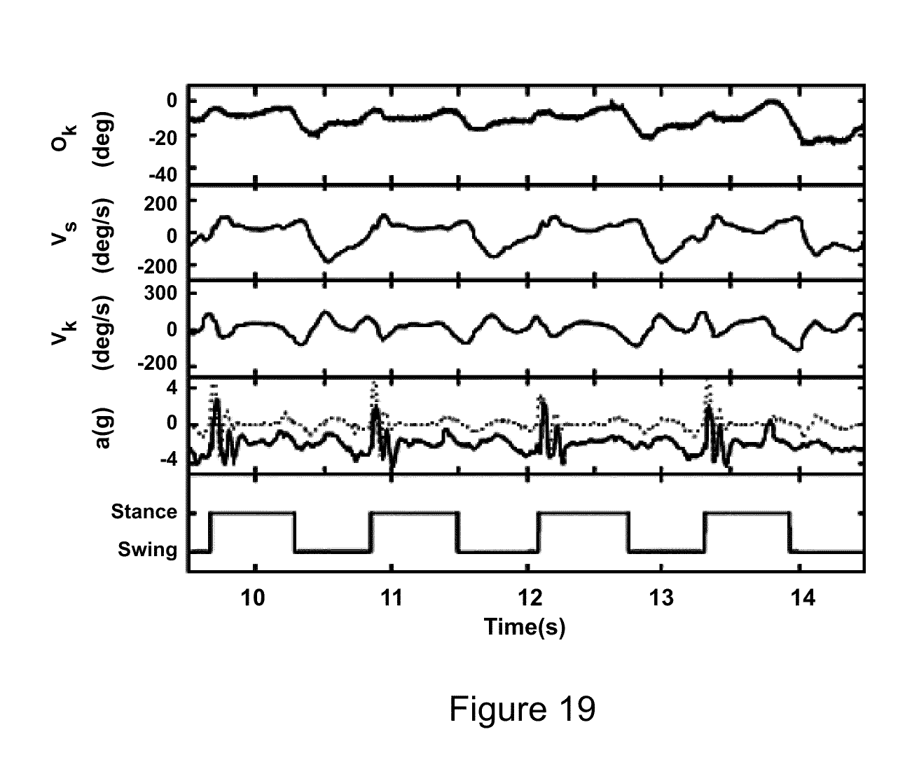

That window, as well as a window for TO, is defined carefully to have efficient detection of the events. Once the time windows are defined, simple criteria are checked against the incoming signals to identify the gait events. The characteristics of the various signals used to window the data and detect events are depicted in FIGS. 18 and 19 for healthy and stroke users respectively.

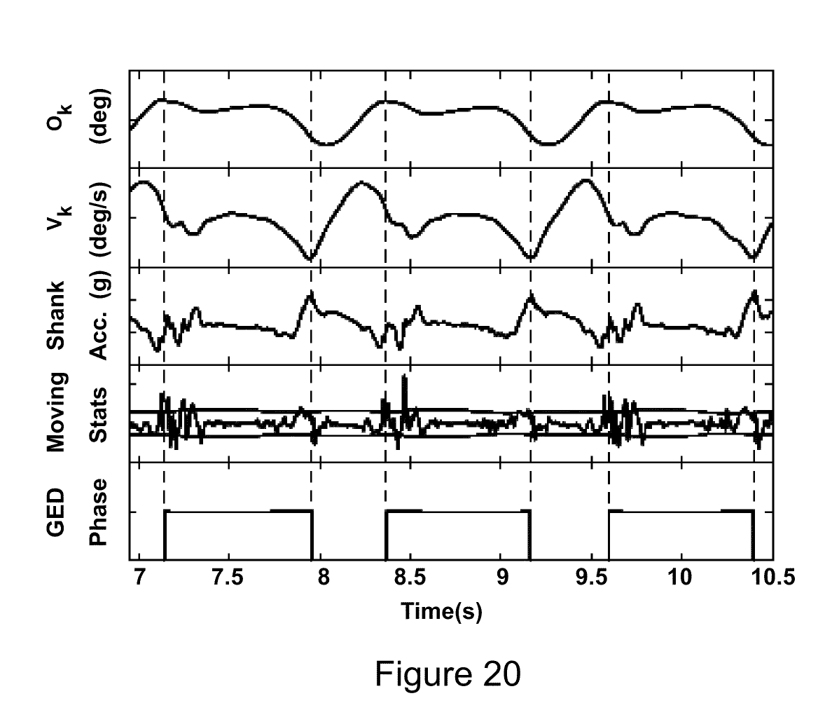

FIG. 20 demonstrates the correlation between the kinematic data and the initial contact and toe off events as detected by brace assembly 100. A starting time and an ending time for the IC time window and the TO time window are be defined with each stride. While the general timing of these windows will be consistent, it will adapt to changing gait patterns over time due to the constant calibration of the system. The starting time to look for an IC event is defined shortly after a minimum in the shank gyroscope signal. The duration of the window is 20% of the last stride length, at which point the ending time is defined. For TO, the algorithm predicts when the next TO should occur by using the last stride length and the last knee velocity minimum time. It starts looking for a TO event at 90% of the last stride length time after the last knee velocity minimum. The duration of the TO window is also 20% of the stride length, at which time the ending of the TO window is marked. With the timing windows defined at each time step, the algorithm then looks for the events. Gait events are detected by checking incoming signals against specific criteria. If the end of either window arrives without detection of the proper event, the end of the window is marked as the event. This prevents any highly irregular strides from impairing the effectiveness of the GED algorithm, essentially removes the effects of strides that are statistical outliers. The algorithm searches for an initial contact first. The criteria for IC are checked against the moving average signal from accelerometer 136 and the standard deviation bounds that were calculated in Stats Subsystem 230.

Referring back to FIGS. 18 and 19, in typical gait of healthy and stroke users, is there is a sharp spike in the horizontal acceleration signal at IC. This is one of the few kinematic characteristics that is common between most healthy and stroke users. The amplitude of this spike will vary greatly, however, the slope of the signal at IC is consistently larger than at other times during the gait pattern. At times when the slope of the horizontal accelerometer signal is large, the moving average of the increments will move outside the standard deviation bounds that are set. When this happens inside the IC window, the event is marked and the system no longer looks for IC, but begins to look for the pre-swing phase. In the event that the moving average does not exceed the standard deviation bounds inside the IC window, the event will either be marked at the end of the IC window or when the first or second pressure sensor signals exceed their threshold value of 0.1. Whichever of these backup events that occur first will be marked as the assumed IC. This redundancy of triggers to mark the IC event makes it difficult to miss a stride completely although slight, usually negligible, errors in the timing of the event may exist.

The pre-swing phase is a period of time shortly before TO. During this phase the user's heel is off the ground, preparing to swing the leg. The detection of this phase is currently done purely on pressure sensors 138. Because of this, the placement of the pressure sensors 138 under the foot will greatly affect the detection accuracy. This phase is marked when the signal from the third set of pressure sensors 138 decreases below the threshold value of 0.1. This phase is detected most reliably when the option to use the GED phase detection in the software has been selected. If pressure sensors 138 are being used to detect events, this event to mark pre-swing is often taken as TO.

As has been discussed, often times the first and last set of pressure sensors do not accept enough load to trigger a signal. In these cases, IC is detected late and TO is detected early by pressure sensors 138. The early detection of TO is the same event that the GED method uses to mark the pre-swing phase. Currently, this phase is marked for research purposes only and does not affect the performance of locks 114, 116. If the phase is missed, locks 114, 116 still disengage appropriately at TO. The detection of TO comes from the knee velocity signal. There is a consistent minimum in knee velocity (thigh-knee gyroscope signals) around TO (FIGS. 18 and 19).

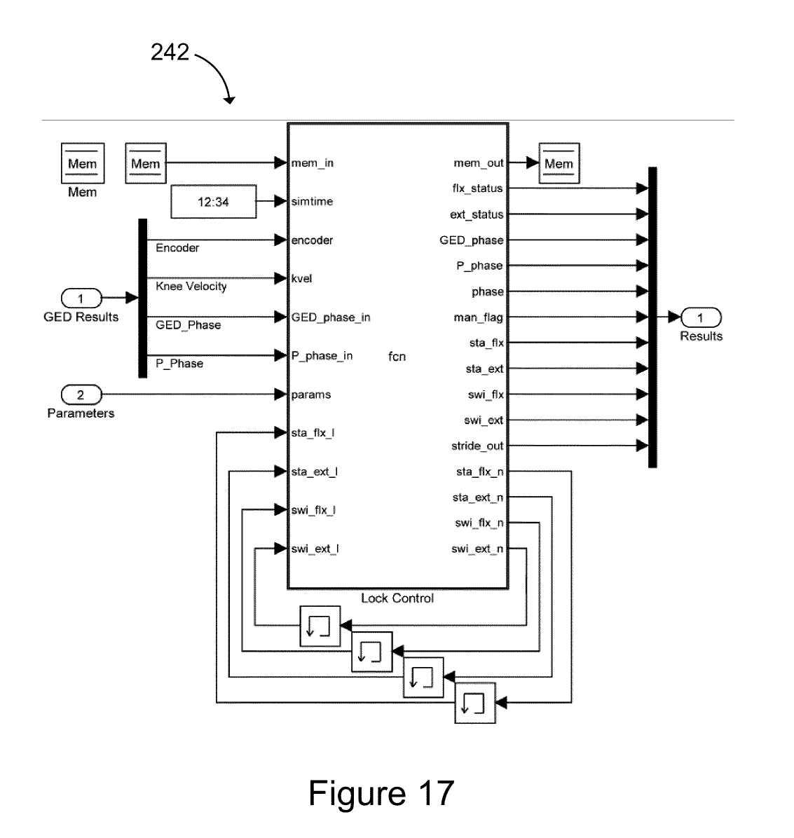

As discussed above regarding Stats Subsystem 230, this minimum is detected by checking various criteria against the knee velocity signal. In Detection Subsystem 240, the same criteria are checked, but only once inside the TO window. If a knee velocity minimum is detected inside the TO window, the event is marked and the system no longer looks for TO, but begins looking again for IC. In the case that a knee velocity minimum matching the necessary criteria is not found in the TO window, the end of the window is marked as the event. From the detected events, a new signal is created for use by Lock Control Subsystem 242. shown in FIG. 17, in the form of a square wave. This new signal, simply called the GED phase, and the phase signal generated by pressure sensors 138 are the two options of controlling locks 114, 116. In the GED phase signal, values of 1, 0.9, and 0 represent the stance, pre-swing, and swing phases respectively. With the detection of events completed, the system now uses this information to control the locking mechanism.

The results from the Detection Subsystem 240, shown in FIG. 21, and the collection of adjustable gains are input into Lock Control Subsystem 242, shown in FIG. 17. Lock Control Subsystem 242 is essentially one embedded Matlab function that controls the output to locks 114, 116. The outputs of Lock Control Subsystem 242 are all written to the data file once the collection has been stopped.

In the embedded function, the first step is to determine which lock settings are enabled. Different lock angles can be set for flexion and extension in both swing and stance. This gives four different controls combining for 16 different ways to lock brace assembly 100. To maintain the most efficient code processing possible, only one numerical value is passed. This value can be any integer between 0 and 15, representing the 16 different control strategies. Any lock 114, 116 that is enabled (has its box checked) gets a value of 1, while being disabled (has its box unchecked) gets a value of 0. A combination of these values in a binary format gives only one value to pass to the target. Once passed to the target computer, the value updates in real time which locks 114, 116 are enabled and which locks 114, 116 are not.

There are other small yet vital calculations and adjustments that take place before engaging or disengaging the locking mechanism. One is that the lock and release angles are adjusted for the time delay as discussed in the previous chapter. The time delay value is simply multiplied by the current knee velocity and subtracted from the lock or release angle provided by user 50. The knee angle is then converted from volts to degrees by using slope and offset values. The last step to prepare for the actual lock control is to apply the choice of which event detection method to use as indicated by user 50, GED or pressure sensors 138.

Once the preparatory calculations are finished, the algorithm begins to check to see if either of the locks should be engaged or disengaged. The first check is to determine if user 50 is entering stance or swing on this time step. If he/she is entering stance or swing, both outputs to the locks are shut off by default so that the settings for the previous phase do not carry over into the new phase. The criteria for this is that the phase signal, whether GED or pressure sensors 138, has a value change from the last sample greater than 0.1. If this happens, it is certain that either stance or swing has been entered at the current time step. If a change of less than or equal to 0.1 occurs, then either there is no change in the phase or pre-swing has been entered, in which case the previous controls will still hold. While the code that checks the knee angle against locking conditions in each phase is ultimately the core control component, it is performed in a simple and efficient way. An exemplary code below shows the code checking for stance phase flexion lock conditions. The same code is repeated for the other locks, substituting the correct phase, locks, and angles for each as required.

```
Initialize FlxStatus = FlxStatus Last
IF phase = 1
      Check
      IF kneeangle > StaFlxRel
            Set FlxStatus = 0
ELSE IF FlxStatus Last = 0
      Check
      IF kneeangle <= StaFlxLock & . . .
            StaFlxEnabled = 1
            Set FlxStatus = 1
            Set StaFlxCnt = StaFlxCnt +1
END
```

In the code above, FlxStatus is the status the flexion lock 114 takes at the end of the current time step (1 for engaged, 0 for disengaged). The FlxStatusLast value is the status of lock 114 at the last time step. Phase specifies stance or swing with a 1 or 0 respectively. “Kneeangle” is the current knee angle value, “StaFlxRel” for flexion lock 114 in stance. “StaFlxEnabled” specifies whether lock 114 is enabled or disabled (a 1 or 0 value respectively) and “StaFlxCnt” is a running count of how many times flexion lock 114 in stance has been triggered. The algorithm first verifies what phase user 50 is currently in. The algorithm then checks to see the status of the appropriate lock 114, 116 at the last time step. If at the last time step the lock 114, 116 was engaged, the algorithm checks to see if lock 114, 116 needs to be disengaged by comparing the knee angle to the release angle. If at the last time step the lock 114, 116 was disengaged, the algorithm checks to see if the lock 114, 116 needs to be engaged by comparing the knee angle to the locking angle and checks to make sure that the specified lock 114, 116 is enabled. If conditions are met in either case, appropriate changes are made to the lock status.

Experimental data is examined to validate the accuracy of the GED algorithm. For healthy user data, ground reaction forces acquired from an instrumented treadmill using a VICON Motion Capture System are used as the standard, in particular the vertical reaction force. For data previously acquired from a stroke user, foot switch data is used as the standard. The foot switch signal shows a delay of only one sample at 1080 Hz when compared to the force plate data and as such, is a reliable standard. While the healthy user data is collected on the treadmill at 2 mph, the stroke user data was collected at free speed over ground while user 50 walked in a circle of a 2.5 m radius. All data from the knee brace is sampled at 600 Hz and the force plate data is captured at 1080 Hz. Down sampling the force plate data in post processing allowed for accurate comparisons of the data from two systems.

Example

Data from one healthy user and one stroke patient user were examined. The healthy user was a 27 year old male with an average stride time of 1.236 seconds. The stroke patient user was a 63 year old male who suffered right hemiparesis due to a stroke seven years prior to testing and whose average stride time was 1.443 seconds. Error in Gait Event Detection using GED Algorithm is shown in Table 1, below.

TABLE 1

| | | Maximum | Minimum | | |
|---|---|---|---|---|---|
| | Event | Error | Error | Mean Error | STD Error |
| Healthy | Initial | 8.24% | 0.07% | 4.02% | 2.38% |
| | Contact | | | | |
| | Toe Off | 6.82% | 0% | 0.95% | 2.25% |
| Stroke | Initial | 2.40% | 0.14% | −0.54% | 1.50% |
| | Contact | | | | |
| | Stroke | 8.91% | 1.63% | 6.11% | 1.54% |

Statistically, the error in detecting events was within an acceptable range for gait training. The algorithm detected initial contact more accurately for the stroke user and toe off more accurately for the healthy user. The TO error for the stroke user data was likely less than the calculations performed here suggest. The foot switches are only capable of signaling TO exactly on time or early. So if the TO results for the stroke user were compared to actual force plate data, the errors would only decrease.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. A controllable brace assembly comprising:
a thigh brace having a thigh gyroscope attached thereto;
a shank brace having a shank gyroscope and a shank accelerometer attached thereto;
a controllable knee lock coupling the thigh brace to the shank brace, the knee lock having an encoder attached thereto;
at least one pressure sensor for attachment to the sole of a user's shoe; and
a controller configured to receive signals from the thigh gyroscope, the shank gyroscope, the encoder, the shank accelerometer, and the at least one pressure sensor, and to transmit a first signal set to engage or disengage the controllable knee lock based on the received signals.

2. The controllable brace assembly according to claim 1, wherein the controllable knee lock comprises a gear plate and a first locking pawl operatively coupled to the controller to engage or disengage the first locking pawl to or from the gear plate.

3. The controllable brace assembly according to claim 2, wherein the gear plate comprises a first plurality of ratchet teeth aligned to allow the first locking pawl to engage one of the first plurality of ratchet teeth in a first direction and to ride over the first plurality of ratchet teeth in a second direction.

4. The controllable knee brace assembly according to claim 3, further comprising a second locking pawl operatively coupled to the controller to engage or disengage the second locking pawl to or from the gear plate.

5. The controllable brace assembly according to claim 4, wherein the gear plate further comprises a second plurality of ratchet teeth aligned to allow the second locking pawl to engage one of the second plurality of ratchet teeth in the second direction and to ride over the second plurality of ratchet teeth in the first direction.

6. The controllable brace assembly according to claim 1, wherein the controller is configured to transmit a second signal set, different from the first signal set, to engage or disengage the controllable knee lock based on the received signals.

7. A method of controlling the operation of the controllable brace assembly of claim 1 comprising the steps of:
(a) coupling the controllable brace assembly to a user's leg;
(b) allowing the user to take several steps, thereby developing a gait cycle; and
(c) automatically controlling operation of the controllable knee lock based on information transmitted to the controller during step (b).

8. The method according to claim 7, further comprising the steps of:
(d) allowing the user to change the gait cycle; and
(e) automatically changing operation of the controllable knee lock based on the change in the gait cycle.

9. The method according to claim 7, wherein step (c) comprises the step of locking the user's knee at a prescribed angle during part of the gait cycle.

10. A controllable brace assembly comprising:
a knee, ankle, foot orthosis comprising a knee lock;
a plurality of sensors coupled to the knee, ankle, foot orthosis;
a first lock configured to releasably lock the knee lock based on data provided by the plurality of sensors.

11. The controllable brace assembly according to claim 10, wherein the first lock is configured to release the knee lock based on data provided by the plurality of sensors.

12. The controllable brace assembly according to claim 10, wherein the first lock is configured to releasably lock the knee lock in a flexion direction.

13. The controllable brace assembly according to claim 12, further comprising a second lock configured to releasably lock the knee lock in an extension direction based on data provided by the plurality of sensors.

14. A method of controlling the operation of the controllable brace assembly of claim 10 comprising the steps of:
(a) coupling the controllable brace assembly to a user's leg;
(b) allowing the user to take several steps, thereby developing a gait cycle; and
(c) automatically controlling operation of the controllable knee lock based on information transmitted to the controller during step (b).
